(19) United States
(12) Patent Application Publication
(10) Pub. No.: US 2005/0245901 A1
(43) Pub. Date: Nov. 3, 2005

(54) GOD Benign Prostatic Hyperplasia (BPH) system to replace a foley catheter, and prevent prostate cancer

(76) Inventor: Herbert R. Floyd, New York, NY (US)

Correspondence Address: HERBERT R. FLOYD, P. O. BOX 286-779, NY CITY, NY, 10128, US

(21) Appl. No.: 11/173,347

(22) Filed: Jul. 1, 2005

Related U.S. Application Data
(63) Continuation-in-part of application No. 08/390,484, filed on Feb. 17, 1995, now abandoned.

(51) Int. Cl. A61M 27/00

(52) U.S. Cl. 604/544

(57) ABSTRACT

A Benign Prostatic Hyperplasi (BPH) 12.0 cm length tube, thereby made of 100% latex-free Silicone, and Rx required for reducing the risk of urinary tract infections. This BPH tube is designed for allowing water to flow from the bladder excluding a Foley Catheter, and leg-bag. When complete blockage occurs, the BPH tube is loaded onto a latex-free threaded bar element for being inserted through a reproductive organ of a male, and moved along in constant frictional contact via his Urethra. The tube is set 2.0 mm from the base of the bladder, and its opposite end is about 1.0 cm beneath an overgrowth of prostate tissue, which pushes against the Urethra that is now opened, and protected by the BPH tube. This can keep the Urethra opened for 120 years, since a doctor can set the tube in place via an ULTRA SOUND.

### BACKGROUND OF THE INVENTION

1. Field of the Invention

The present invention relates to Prostate Cancer, and specifically to a Latex free flexible BPH tube. This tube is defined by a diameter, which is shaped to conform to the contours of one Urethra, a tube that carries water out of the Bladder and through the Penis. Further, the BPH, or benign prostatic hyperplasia tube thus comprises three lengths: 7.0 cm lengths are for mild BPH symptoms, 10.0 cm tubes are for moderate BPH symptoms, and 11.0 cm−12.0 cm lengths are inserted, if complete blockage occurs. These tubes each of which can be inserted through the Penis via the Urethra, and moved along in frictional contact using a loading tool to set the BPH tube in place adjacent to the Bladder, and an Enlarged Prostate. The BPH tube protects the Urethra from the Enlarged Prostate, which obstructs water from flowing via the Bladder, and through the Urethra. The Prostate surrounds part of the Urethra, and tends to grow larger with age, thereby pressing against the Bladder, thus of which can squeeze the Urethra. This can slow down water flow, or block water from flowing through the Urethra. As the loading tool is incorporated for loading the BPH tube, it is further used for the removal of the BPH tube from the Urethra. The BPH tube removal is accomplished as the loading tool is inserted through the BPH tube, as the BPH tube is preventing the Prostate from squeezing the Urethra. At this position, a balloon is inflated, only to serve as a fastening device, whereby to fasten the exterior surface of the loading device to the interior surface of the BPH tube. This will allow the loading tool including the BPH tube to be removed via the Urethra, and through the Penis. Exterior screw threads about a front end of a loading tool can be used to fasten the tool to the rearward, and interior treaded surface of the BPH tube for the installation and removal thereof. A doctor can view the installation or the removal of the BPH tubes via an Ultra Sound.

2. Description of the Prior Art

Many prior art devices and BPH Medications exist to attempt to prevent the use of a Foley catheter, since the catheter is defined by a leg bag, and a flexible tube inserted through a Penis of a male to deliver fluids into, or withdraw fluids from the body. There are two main types of BPH Medications used. One type relaxes muscles near the Prostate, while the other type shrinks the Prostate gland.

As these Medications are worthwhile, you must continue to take the first type of medication while the catheter is in place until the medication relieves pressure, and let water flow more freely. Taking the second type of medication for at least six months to one year can increase water flow, and reduce symptoms. It can also cause Decreased interest in sex, Trouble getting or keeping an erection, and smaller portions of semen with ejaculation. Consequently, it is still unclear whether the drug lowers the risk of dying from Prostate cancer.

GOD BPH tube is a revolutionary 21^{st} Century reality for which a catheter has been excluded from the Urethra, and a leg bag is no longer needed. Besides, a slot is provided in the right hand side of the BPH tube, seeing that the Prostate is a reproductive system, and helps make semen. The semen carries sperm from two Testicles through the Penis via the Prostate, and its Seminal vesicle member, which includes a tube that branches out from the Urethra. This Seminal vesicle tube is for sending sperm on a short course of travel in a brief space of time, however, through the aforementioned slot, including the BPH tube, the Urethra, and leaves the body through the Penis when a man ejaculates. With respect to the BPH tube, a man can empty his Bladder accordingly, when he passes fluids which are more desirable than using a catheter to drain fluids from the Bladder and into a leg-bag. As a rule, the installation of the BPH tubes each of which can be performed, while the doctor is viewing a monitor with respect to the “ULTRA SOUND.”

### SUMMORY OF THE INVENTION

Accordantly, it is a principle object of the present invention to produce one flexible tube having a diameter which is shaped to conform to the contours of the Urethra, however, its length thereof should start at the base of the Bladder, and end just below the enlarged Prostate. This benign prostatic hyperplasia (be-NINE prah-STAT-ikHY-per-PLAY-zha) BPH tube will allow a man to pass his water freely, and in such a manner that the use of a Foley catheter will be eliminated. This BPH tube can be installed in the Urethra and left in place for an indefinite time. Now, the enlarged Prostate gland will surround the Urethra including the BPH tube, which will prevent the Prostate from affecting the flow of water that proceeds from the Bladder. Notwithstanding, to accomplish the foregoing, and other objects, a BPH tube to prevent BPH surgery, a spinal block used to numb the area, and the catheter to drain water for a period of time after the surgery procedure, thus according to the present invention comprises: a Benign Prostate Hyperplasia BPH tube set via the Urethra located at the base of the Bladder adjacent to an Enlarged Prostate glan for preventing BPH symptoms, PBH surgery and Prostate Cancer with respect to a first conventional tube having a length starting at the Prostate and ending at the base of the Bladder, thereby, receiving fluids via the first tube, the length indicates mild symptoms, the BPH tube prevents symptoms, a second conventional tube including a longer length starting at the Prostate, and ending at the base of the Bladder thereby receiving the fluids from the Bladder, the length is defined by moderate BPH symptoms, and a third conventional tube including the longest length starting at the Prostate, and ending at the base of the Bladder for withdrawing the fluids from the Bladder, when complete blockage occurs, the BPH tube prevents blockage, a first loading tool having a conventional inflatable device for causing stability to the first, second, and third BPH tubes before removing the BPH tubes from the Urethra, where upon installation, the inflatable device is deflated, a second loading tool having an exterior threaded surface at its front end, and engaging three interior threaded surfaces in three rearward openings about the BPH tubs, whereby to load or unload the BPH tubes through the Urethra. The BPH tubes thereby, are shaped interiorly to fit the exterior surface of the first loading tool, the first loading tool extends through the front of the BPH tubes. The first loading tool has a hole below the inflatable device for inflating the inflatable device to secure the BPH tubes to the first loading tool. The first loading tool thereby has a larger diameter at its rearward portion than its front portion, the larger diameter has a ledge to mate with the BPH tubes at the openings. The inflatable device is thereby fixed between the front of the first loading tool and the ledge, the rearward openings of the BPH tubs fit the ledge. The front of the first loading tool whereby extends through the front of the BPH tubes, the front of the first loading tool has a pointed portion. The pointed portion has a system for, thereby penetrating an opening of the Urethra, the BPH tubes are set within the Urethra to open the Prostate. The Prostate gland thereby, is opened via the BPH tubes, the fluids are allowed to flow from the Bladder, through the BPH tubes, and via the Urethra. The Prostate is a male reproductive system, whereby making semen for carrying sperm from two testicles to the Prostate via a Seminal vesicle member. The Seminal has a tube joining the Urethra for carrying the sperm through the Seminal tube, three slots of the BPH tubes, and through the Urethra. When at least one BPH tube is set in the Urethra having its front surface near the Bladder, its rearward opening is set erectly beneath the Prostate gland, and the slot is adjacent to the Seminal tube. The BPH tubes are thereby set in the Urethra via a mid Prostate location for the fluid to flow, the Prostate comprises the Urethra including the BPH tubes. The BPH tubes are left in place via the Urethra for an indefinite time, and without the use of a Foley catheter. The BPH tubes are defined by a system for allowing a user to empty his Bladder accordingly. The BPH tubes are set in the Urethra by the use of a clear BPH numbing medication. The slots are, thereby incorporated in the first BPH tube, the second BPH tube, and the third BPH tube for receiving the sperm from the Seminal tube. The fluids, thereby having water products, the fluids are made by a user's kidneys, stored in the Bladder and leaves the body via the Urethra. The BPH tubes are installed and set in place by a Doctor specializing in the urinary organs in females, thereby including the urinary and sex organs in males. The PBH tubes, whereby eliminate mild-to-moderate BPH symptoms, BPH Medications the catheter and BPH surgery, when the BPH tubes each of which is set in place. The BPH surgery is, thereby unwanted by Millions of American men, the Prostate Cancer is prevented, when the BPH tubes are installed and set in place via an Ultra Sound.

### BRIEF DESCRIPTION OF THE DRAWINGS

Further objects and advantages given herewith about the present invention will become apparent, thereby the drawings with respect to the preferred embodiments concerning the invention, and the description thereof;

FIG. 1 is a perspective view of three BPH tubes each of which can prevent BPH symptoms defined by mild, moderate, and complete blockage through the Urethra;

FIG. 2 is a side view of two loading tools which are diverts from one another, when loading and unloading BPH tubes via an inflatable device and a helix system;

FIG. 3 is a perspective view of the loading tool defined by the inflatable system;

FIG. 4 is a perspective view of the other loading tool having the helix system;

FIG. 5 is a perspective view of the inflatable device including its mechanism;

FIG. 6 is a side view about this helix system for, only unloading the BPH tubes;

FIG. 7 is a side view of one BPH tube loaded on the inflatable loading system;

FIG. 8 is a perspective view of a catheter having a pointed end for entering the Urethra, and moved along in frictional contact thus in opposition to the Bladder, a balloon is inflated to prevent the removal of the catheter from the Bladder;

FIG. 9 is a view of a Foley (leg bag), which receives water from the Bladder via the catheter that is positioned in the Urethra, and is connected to a user's leg; A Flowchart is provided in FIG. 10

### DETAILED DESCRIPTION OF THE PREFERRED EMBODIMENTS

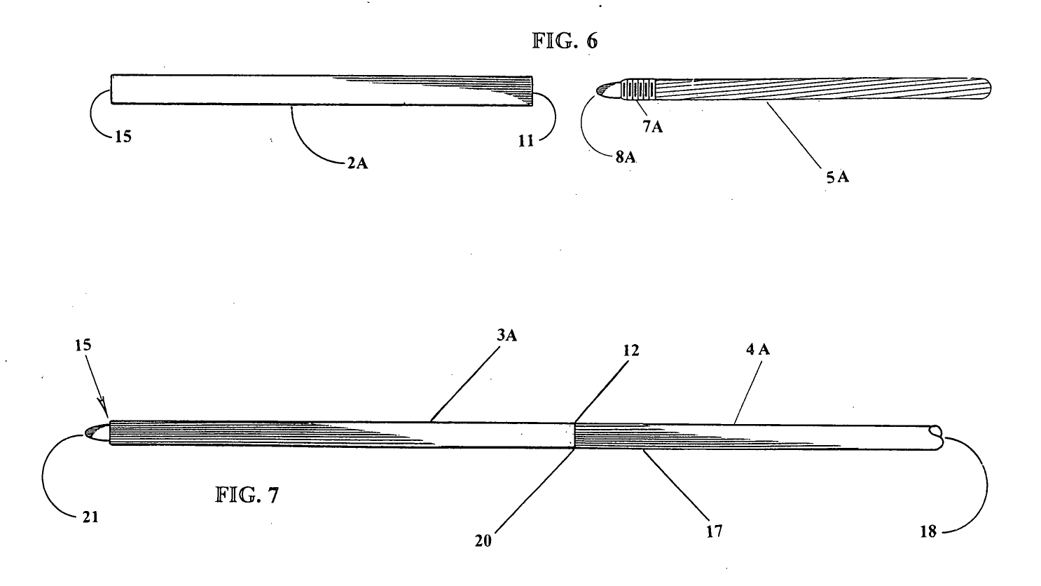

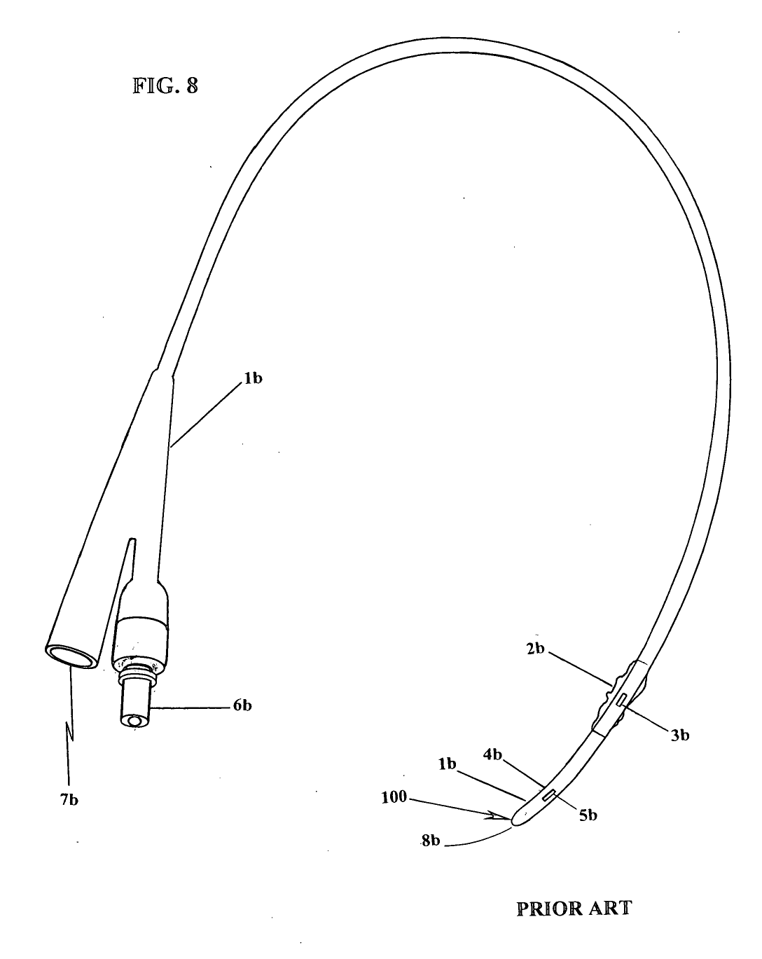

Prostate enlargement in elderly males is the most common benign (noncancerous) tumor like growth in men. This enlargement of the prostate is called BPH for benign prostate hyperplasia. The prostate gland surrounds the urethra at the base of the bladder. When it grows, the prostate blocks the urethra. Symptoms of BPH include weak water stream, dribbling, feeling like the bladder has not emptied completely, and difficulty starting urination. Other symptoms include getting up frequently both at night and during the day to urinate. Complete urinary blockage or severe pain with urination require immediate attention. If the symptoms are mild, no treatment may be necessary for BPH. If the symptoms are moderate, alpha blocker medications (Cardura, Flomax and Hytrin) may provide relief. Proscar and Duagen works by making the prostate gland smaller. The herb saw palmetto has not been labeled by the FDA for treatment of BPH, but is commonly used in Europe. If complete blockage occurs, a Foley catheter (FIGS. 6-7) is usually left in place for a few days. Surgery is often needed for more severe prostate blockage symptoms. TURP is the prostate surgery for BPH which is done through the urethra. Techniques using heat, microwave and laser to remove the prostate blockage are also being studied. Stay away from alcohol, caffeine beverages (coffee, tea, colas), and medicines which make the problem worse. Decongestants, antihistamines, and some prescription medicines can also make the problem worse. You should be seen right away or go to the emergency room for these symptoms: Increased pain with urination or being unable to urinate, severe abdominal pain, vomiting, a high fever, or fainting.

The prostate is a small gland in men. It is part of the male reproductive system. The prostate is about the size and shape of a walnut. It sits low in the pelvis, below the bladder and just in front of the rectum. The prostate helps make semen, the milky fluid that carries sperm from the testicles through the penis when a man ejaculates. Since the prostate surrounds part of the urethra, a tube that carries water out of the bladed and through the penis, this can be a source of problems as a man ages. Consequently, the prostate tends to grow bigger with age and may squeeze the urethra (no photos are available at this present time), notwithstanding, a tumor can make the prostate bigger. These changes, or an infection, can cause problems passing water. Sometimes men in their 30s and 40s may begin to have these urinary symptoms and need medical attention. For others, symptoms are not noticed until much later in life. Growing older raises your risk of prostate problems. The three most common prostate problems are: Infection (prostatitis), Enlarged prostate (BPH, or benign prostatic hyperplasia), and Prostate cancer. One change does not lead to another. For example, having prostatitis or an enlarged prostate does not rise your chance of prostate cancer. It is also possible for you to have more than one condition at the same time.

Most men have prostate changes that are not cancer. Abnormal findings from any of these following tests can help diagnose a problem and suggest the next steps to take: DRE (digital rectal exam)—a test to feel the prostate, PSA (prostate-specific antigen) test—a blood test, and Biopsy—a test to check for cancer.

With respect to the above modification, prostatitis (pronounced “PRAH-stuh-TYE-tis”) is an inflammation or infection of the prostate gland. It affects half of all men at some time in their lives. Having this condition does not increase your risk of any other prostate disease. Prostatitis Symptoms are: Trouble passing water or pain when passing water, A burning or stinging feeling when passing water, A strong frequent urge to pass water, even when there is only a small amount of water, Chills and fever, Low back pain or body aches, Pain low in the belly, groin, or behind the scrotum, Rectal pressure or pain, Urethral discharge with bowel movements, Genital and rectal throbbing, Sexual problems and loss of sex drive, Blocked water, and Painful ejaculation (sexual climax). Prostatitis is not contagious. It is not spread through sexual contact. Your partner cannot catch this infection from you. Several tests, such as DRE and a water test can be done to see if you have prostatitis. Getting the right diagnosis of your exact type of prostatitis is the key to getting the best treatment. Even if you have no symptoms, you should follow your doctor's suggestion to complete treatment.

There are four types of prostatitis: The first is acute bacterial prostatitis. This infection comes on suddenly (acute) and is caused by bacteria. Symptoms include severe chills and fever. There is often blood in the water. You must go to the doctor's office or emergency room for treatment. It's the least common of the four types, yet it's the easiest to diagnose and treat. Most cases can be cured with a high dose of antibiotics, thereby taken for 7 to 14 days, and then lower doses for several weeks. You may also need drugs to help with pain or discomfort.

Chronic bacterial prostatitis, also caused by bacteria, this condition doesn't come on suddenly, but it can be bothersome. The only symptom you may have is bladder infections that keep coming back. The case may be a defect in the prostate that lets bacteria collect in the urinary track. Antibiotic treatment over a longer period of time is best for this type. Treatment lasts from 4 to 12 weeks. This type of treatment clears up about 60 percent of cases. Long-term, low-dose antibiotics may help relieve symptoms in cases that won't clear up.

Chronic prostatitis or chronic pelvic pain syndrome disorder is the most common but least understood form of the disease. Found in men of any age from late teens to elderly, its symptoms go away and then return without warning. There can be pain or discomfort in the groin or bladder area. There are several different treatments for this problem, however, based on your symptoms. These include antibiotics and other medicines, such as alpha-blockers. Alpha-blockers relax muscle tissue in the prostate to make passing water easier.

Asymptomatic inflammatory prostatitis is, such that you usually don't have symptoms with this condition. It is often found when your doctor is looking for other conditions like infertility, or prostate cancer. If you have this problem, often your PSA test will show a higher number than normal. It does not necessarily mean that you have cancer. Men with this condition are almost all ways given antibiotics for 4 to 6 weeks, and then have another PSA test.

We know that BPH stands for benign prostate hyperplasia (pronounced “be-NINE prah-STAT HY-per-PLAY-zha”). Benign means “not cancer,” and hyperplazia means too much growth. The result is that the prostate becomes enlarged. BPH is not linked to cancer and does not raise your changes of getting prostate cancer—yet the symptoms for BPH and prostate cancer can be similar. BPH symptoms almost all ways start after the age of 50. They can include symptoms in the same manner, as set forth in the foregoing with respect to the above modification, thus of which include stopping and starting again several times while passing water. At its worst, BPH can lead to: A weak bladder, backflow of Fluid containing water and waste products, which is made by the kidneys, stored in the bladder, and leaves the body through the urethra. This Fluid can cause bladder or kidney infections, complete block in the flow of Fluids, and kidney failure. BPH affects most men as they get older. It can lead to urinary problems like those with prostatitis. By age 60, many men have signs of BPH. By age 70, most all men have some prostate enlargement.

The prostate starts out about the size of a walnut. By the time a man is 40, it may have grown slightly larger, to the size of an apricot. By age 60, it may be the size of a lemon.

As a normal part of aging, the prostate enlarges and can press against the bladder and the urethra. This can slow down or block water flow. Some men might find it hard to start a water stream, even though they feel the need to go. Once the water stream has started, it may be hard to stop. Other men may feel like they need to pass water all the time or are awakened during sleep with the sudden need to pass water.

Early BPH symptoms take many years to turn into bothersome problems. These early symptoms are a cue to see your doctor. About half the men with BPH eventually have symptoms that are bothersome enough to need treatment. BPH can not be cured, but drugs or surgery can often relieve its symptoms. BPH symptoms do not always grow worse.

There are three ways to manage BPH: Watchful waiting (regular follow-up with your doctor), Drug therapy, and Surgery. Men with mild symptoms of BPH who do not find them bothersome often choose this approach. Watchful waiting means getting annual checkups. The checkups can include DREs and other tests. Treatment is started only if symptoms become too much of a problem. If you choose to live with symptoms, these simple steps can help: Limit drinking in the evening, especially drinks with alcohol or caffeine, empty the bladder all the way when you pass water, use the restroom often, and don't wait for long periods without passing water. Some medications make BPH symptoms worse, so talk with your doctor or pharmacist about any medicines you are taking such as: Over-the-counter cold and cough medicines (especially antihistamines, Tranquilizers, Antidepressants, and Blood pressure medicine.

Millions of American men with mild-to-moderate BPH symptoms have chosen prescription drugs over surgery since the early 1990s. There are two main types of drugs used. One type relaxes muscles near the prostate wile the other type shrinks the prostate gland. There is evidence that shows that taking both drugs together may work best to keep symptoms from betting worse.

Alpha-blockers help relax muscles near the prostate to relieve pressure and let water flow more freely, but they don't shrink the size of the prostate. For many men, the drug can improve water flow and reduce symptoms within days. Possible side effects include dizziness, headache, and fatigue.

Five alpha-reductase inhibitor drug consequently known as finasteride, shrinks the prostate. It relieves symoptoms by blocking an enzyme that acts on the male hormone, testosterone, to boost organ growth. When the enzyme is blocked, growth slows down. This helps shrink the prostate, reduce blockage, and limit the need for surgery. Taking this drug for at least 6 months to 1 year can increase water flow and reduce your symptoms. It seems to work best for men with very large prostates. You must continue to take the drug to prevent symptoms from coming back. This drug can cause the following side effects concerning men: Decreased interest in sex, trouble getting or keeping an erection, and smaller amount of semen with ejaculation.

Before we discuss BPH surgery, GOD ALMIGHTY BPH MIRACEL tube will eliminate “Prostate Cancer.” Drug therapy is defined by side effects, which include dizziness, headache, fatigue, Decreased interest in sex, trouble getting or keeping an erection, smaller amount of semen with ejaculation, and it is still unclear whether finasteride lowers the risk of dying from prostate cancer.

BPH “SURGERY” is used when symptoms are severe, or drug therapy has not worked well. There are six types of BPH surgeries each of which is diverts from one another. The first type is TURP (transurethral resection of the prostate), which is the most common surgery for BPH. It accounts for 90 percent of all BPH surgeries. It takes about 90 minutes. The doctor passes an instrument through the urethra and trims away extra prostate tissue. A spinal block is used to numb the area. Tissue is sent to the laboratory to check for prostate cancer. TURP generally avoids the two main dangers linked to other prostate surgeries: Incontinence (not being able to have an erection). The recovery period of TURP is much shorter also.

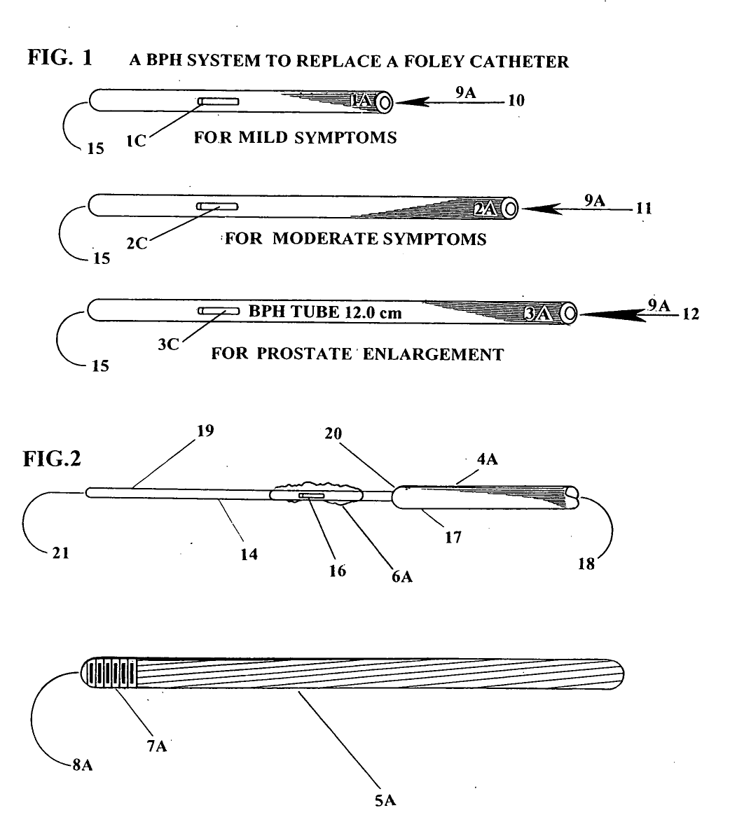

TUIP (transurethral incision of the prostate) is similar to TURP. It is used on slightly enlarged prostate glands. The surgeon places one or two small cuts in the prostate. This relieves pressure without trimming away tissue. It has a low risk of side effects, whereby GOD ALMIGHTY BPH tubes, as shown in FIG. 1, have no risk of side effects. Like TURP, the TUIP treatment helps with water flow by widening the urethra.

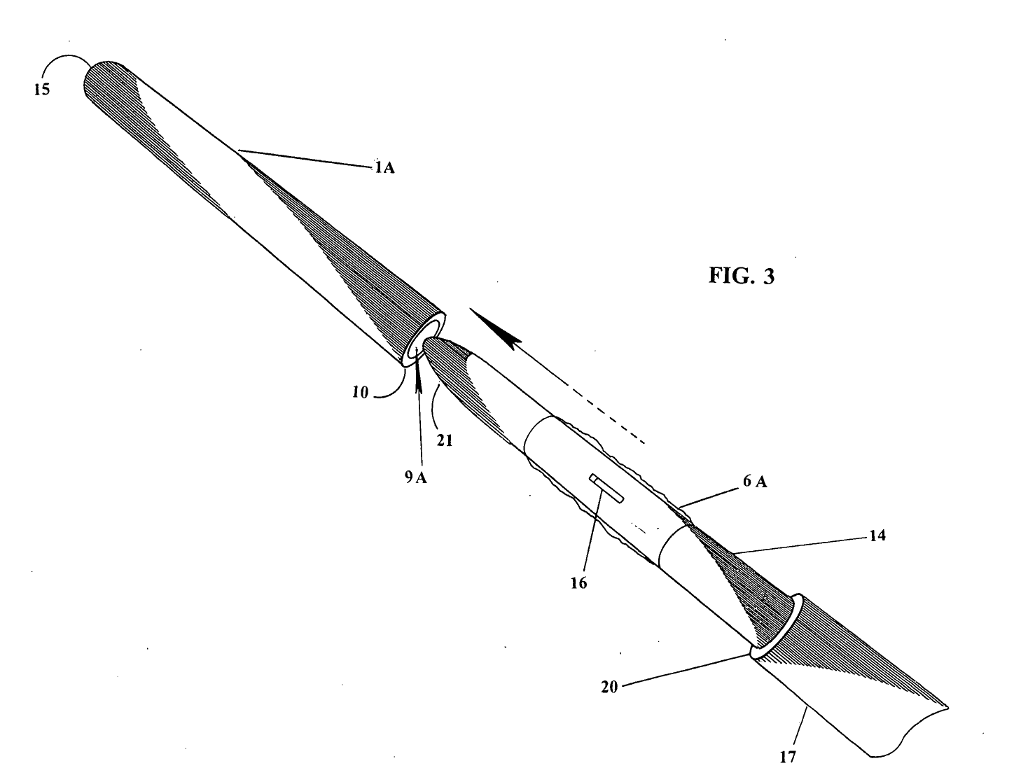

TUNA (transurethral needle ablation) burns away excess prostate tissue using radio waves. It helps with water flow, relieves symptoms, and may have fewer side effects than TURP. The BPH tube 1A, as shown in FIG. 3, provides water flow, eliminates BPH symptoms, and has no side effects. Most men need a catheter 1b, as shown in FIG. 8, however, to drain water for a period of time after the TUNA surgery procedure. Moreover, the catheter 1b (KATH-ih-ter) is a flexible tube used to deliver fluids into or withdraw fluids from the body. The catheter 1b further includes a balloon 2b defined near its front end 8b, which has two holes: 4b on one side, and 5b on the other side. The catheter 1b with respect to its front end 8b includes a solid pointed portion 100, which can penetrate the urethra found at the front end of a user's penis. Where upon penetration, the catheter is moved along in constant frictional contact with the interior surface of the urethra, consequently, in opposition to a user's bladder. As a rule, the pointed portion 100 enters the bladder, whereby the interior surface of the bladder will obstruct the front end 8b of the catheter 1b. Now, a user's doctor may inflate the balloon 2b, thus of which will prevent the catheter from being removed from the bladder. Besides, the two holes 4b-5b will receive water from the bladder, which will flow via the interior portion of the catheter 1b, and through a connecting member 7b formed at the other end of the catheter 1b. The member 7b is adjacent to a device 6b for inflating the balloon 2b, seeing that the doctor connects a syringe (not shown) to the inflating device 6b, and inflates or deflates the balloon 2b via a slotted hole 3b formed in the catheter 1b just beneath the deflated balloon 2b.

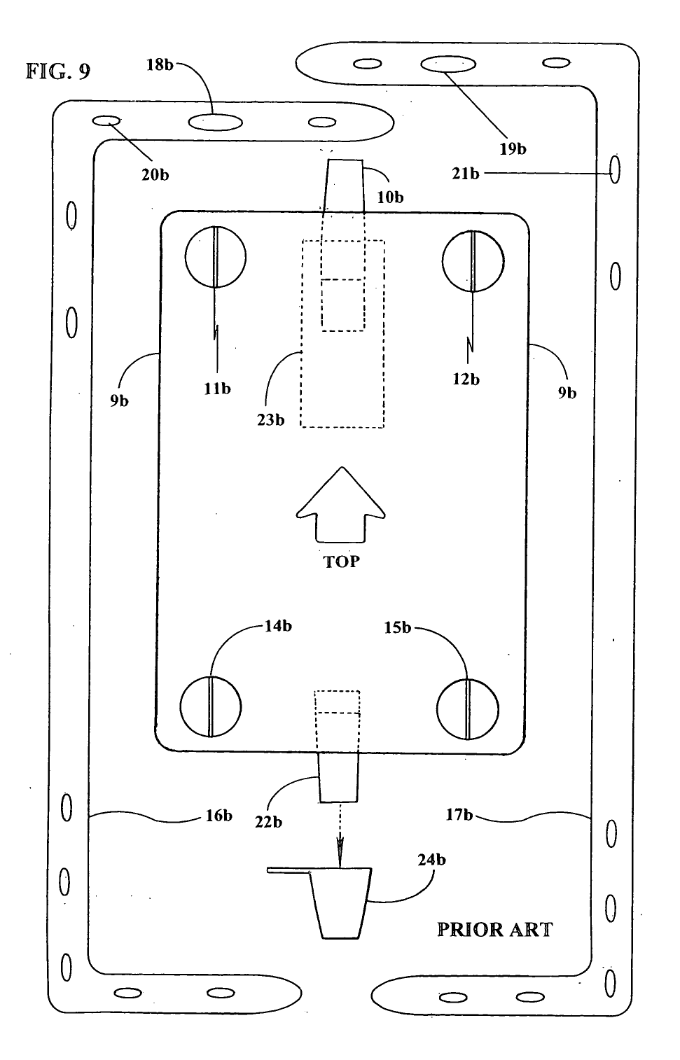

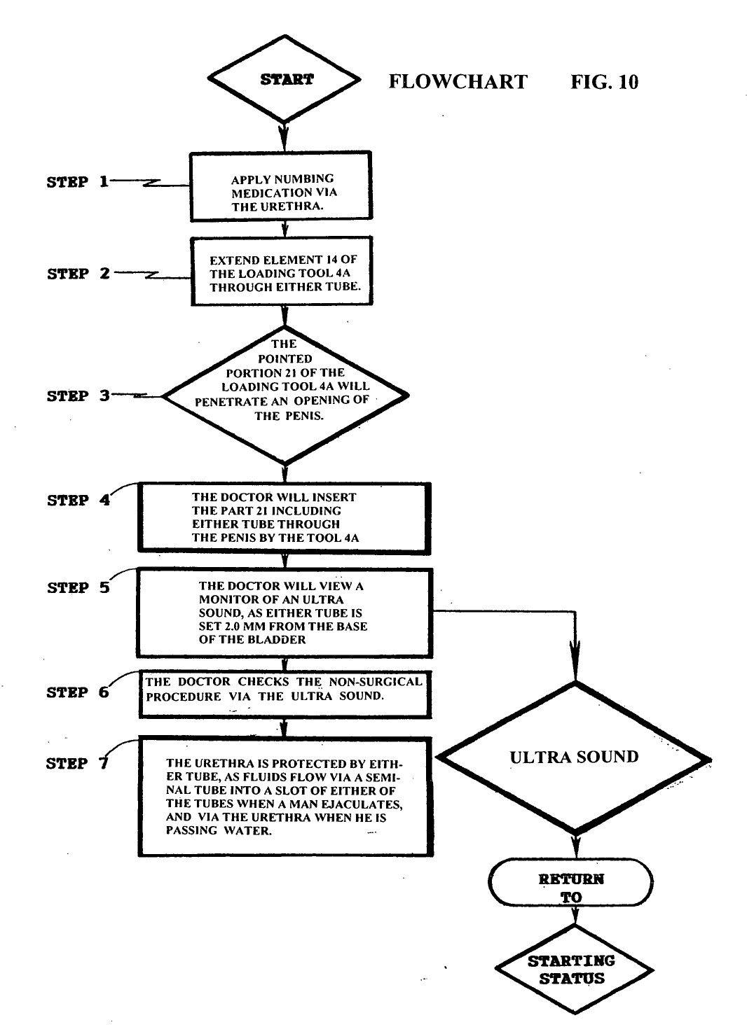

Referring to FIG. 9, a Urinary Leg-Bag 9b has an Inlet valve 10b at the top portion thereof, for connecting to the member 7b about a press fit. The Urinary bag 9b further includes four slits 11b, 12b, 14b and 15b for receiving two straps 16b and 17b. The strap 16b may be passed through the slits 11b-12b, and the strap 17b will be passed through the slits 14b-15b. These straps 16b-17b may now be connected to a user's leg by two button type fastening devices 18b-19b, which fit in either group of holes 20b or 21b in each strap 16b-17b. The Inlet 10b has a Flutter valve 23b which prevents Backflow of fluids, which could cause bladder, or kidney infections. Valve 22b includes a cap 24b to prevent the Urinary Foley leg-bag from leaking.

TUMT (transurethral microwave thermotherapy), thereby using microwaves sent via the catheter 1b to destroy excess prostate tissue. This can be an option for men who should not have major surgery because they have other medical problems.

TUVP (transurethral electroevaporation of the prostate) uses electrical current to vaporize prostate tissue.

Open prostatectomy means the surgeon removes the prostate through a cut in the lower abdomen. This is done only in very rare cases when obstruction is severe, the prostate is very large, or other procedures can't be done. General or spinal anesthesia is used and the catheter 1b remains for 3 to 7 days after the surgery. This surgery carries a higher risk of complications than medical treatment. Tissue is sent to the laboratory to check for prostate cancer.

Prostate cancer means that cancer cells form in the tissues of the prostate. It is the most common cancer in American men after skin cancer. Prostate cancer tends to grow slowly compared with most other cancers. Cell changes may begin 10, 20, or 30 years before a tumor gets big enough to cause symptoms. Eventually, cancer cells may spread (metastasize) throughout the body. By the time symptoms appear, the cancer may be more advanced.

By age 50, very few men have symptoms of prostate cancer, yet some precancerous or cancerous cells are present. More than half of all American men include Some cancer in their prostate glands by the age of 80. Most of these cancers never pose a problem. They either give no signs or symptoms or never become a serious threat to health. A much smaller percentage of men are actually treated for prostate cancer. Most men with prostate cancer do not die from this disease. Nevertheless, in 2003 a Public Health Concern reported, however, that in 2004, approximately 230,110 new cases of prostate cancer will be diagnosed, and 29,900 men will die of the disease, according to the American Cancer Society. Prostate cancer is the second leading cause of cancer deaths of men in the United States, after lung cancer, and the sixth leading cause overall.

With respect to the detailed description, the present invention will be discussed according to the preferred embodiments. Since there are drawings concerning the above brief description of the several views, these views will be referred to by specifying the numbers of the figures. The reference to these drawings shall be defined by different parts by use of reference letters or numerals (preferably the letters). This specification will conclude with twenty new claims, particularly pointing out and distinctly claiming the subject matter, which the applicant regards as his invention.

### First Embodiment

Referring to the drawings, and chiefly to FIGS. 1, and 2, the present invention relates to Prostate Cancer, and specifically to a non-latex flexible BPH tube 1A that combines the latest advancements in intermittent BPH tube comfort, convenience, protection and design. This tube 1A is defined by 100% Silicone, or Silicone coated and being Rx required for reducing the risk of urinary tract infections. This BPH tube (Benign Prostatic Hyperplasia) is available in three lengths for men who use an intermittent BPH tube system as their method of bladder control. These BPH tubes 1A-3A each of which is made with the highest quality standards dedicated to comfort, and an ultra smooth exterior. The BPH tubes 1A-3A are defined by a diameter being shaped to conform to the contours of a user's urethra, a tube for which water is carried from the bladder, and out of the body through the penis. The three aforementioned lengths are such that 7.0 cm lengths are for mild BPH symptoms, the 10.0 cm tubes 2A are for moderate BPH symptoms, and 11.0 cm−12.0 cm lengths are inserted, if complete blockage occurs. These tubs 1A-3A each of which can be inserted through the penis via the urethra, and moved along in constant frictional contact thereby, using a loading tool 4A to set the BPH tube in place, such that your Urologist (yoo-RAH-luh-jist) can view the BPH tube installation on a monitor defined by an ULTRA SOUND system. Consequently, this Ultra Sound system will allow the Urologist to perform the installation accordingly, since he/she, e.g., can see the imprecation of the BPH tube 3A within the urethra. With respect to the Ultra Sound, the doctor can see the front portion of the BPH tube 3A being positioned, and set 2.0 mm from the base of the bladder, whereby the rearward portion of the BPH tube 3A will be seen at least 1.0 cm just beneath, the enlarged prostate.

This treatment will cause a man to pass his water accordingly, and a spinal block is excluded from this procedure, since numbing medication is used before inserting the BPH tubes 1A-3A. This numbing medication is such that a user will not even feel the insertion of the BPH tubes 1A-3A, nor the loading tool 4A.

### The Second Embodiment

A first conventional BENIGN PROSTATIC HYPERPLASIA (BPH) tube 1A is defined by the length of 7.0 cm. Consequently, when the tube 1A is inserted, and its front end 15 is set 2.0 mm from the base of the bladder, the opposite end 10 of the BPH tube 1A is set just beneath the prostate about 1.0 cm. This BPH prostate tube 1A is designed to eliminate mild symptoms. As a rule, the Urologist can view the insertion, including the installation of the BPH tube 1A, while performing a non-surgical procedure by the use of the aforementioned device defined by the ULTRA SOUND.

A second conventional BPH tube 2A having a longer length, which is defined by 10.0 cm, whereby its front end 15 is about 2.0 mm from the base of the bladder. As a result, the rearward end 11 of the BPH tube 2A is set in the Urethra just below the Prostate. Notwithstanding, the doctor can check the installation via a sever case of scrutiny with respect to the ULTRA SOUND system, and this BPH tube 2A is designed to eliminate moderate symptoms.

A third conventional tube 3A having the longest length thereof, and starting about 1.0 cm beneath the prostate, whereby ending at 2.0 mm adjacent to the base of the bladder. This 11.0 cm−12.0 cm BPH tube 3A is inserted through the Urethra, only when complete blockage occurs. Nevertheless, the BPH tubs 1A-3A will prevent complete blockage for one hundred, and twenty years (120 years).

A first loading tool 4A is defined by an inflatable system 6A, which includes a soft lightweight 100% latex-free element for causing stability to the first, second, and third BPH tubes 1A-3A. Since the first loading tool 4A is shaped on its exterior surface to conform to the contours of the interior surface within the BPH tubes 1A-3A, the inflatable element 6A is inflated when the first loading tool 4A is inserted through either of the BPH tubes 1A-3A. As a result, the inflatable element 6A serves as a fastening device, such that the first loading tool 4A is fastened to the inner surface of either of the BPH tubes. Now the BPH tubes 1A, 2A, or 3A can be moved through the Urethra, and set in place by the Urologist via the Ultra Sound. The first loading tool 4A is removed from the BPH tubes 1A-3A, when the inflatable element 6A is deflated via a syringe connecting an inflating, and deflating system 6b.

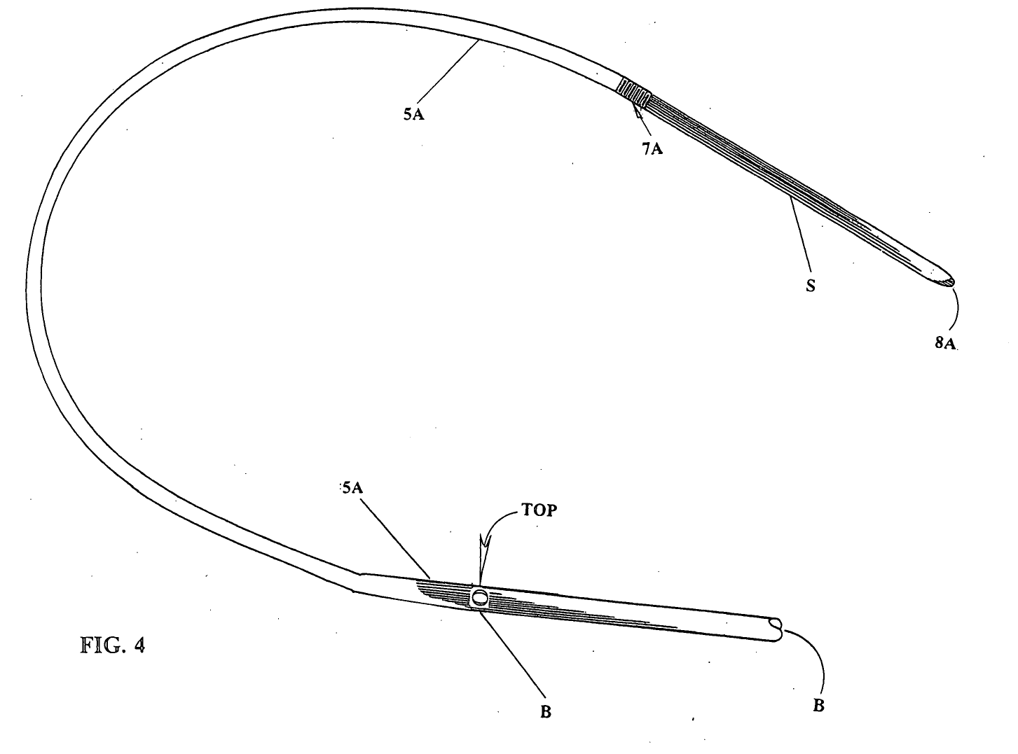

A second loading tool 5A has an outer threaded bar surface 7A formed, below an extended bar surface S for engaging three interior threaded surfaces 9A in three rearward openings 10-12 of the BPH tubes 1A-3A. This is to load and unload the BPH tubes 1A-3A via the urethra, as the slots 1C-3C appear at the right of each tube, as indicated by a mark O on top of handle B of the tool 5A (FIG. 4). As the BPH tubes 1A-3A are shaped interiorly to fit the outer surfaces 14 and S of the tools 4A and 5A, these tools 4A-5A extend through the front 15 of the BPH tubes 1A-3A.

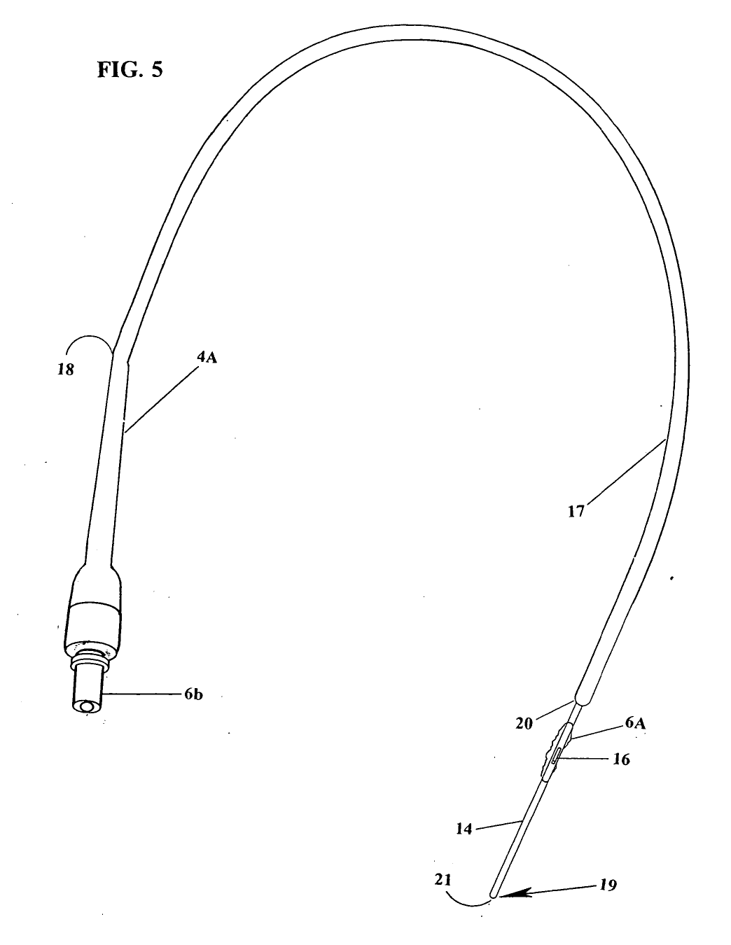

Referring to FIGS. 3, 4, and 5, the first loading tool 4A has a hole 16 below the inflatable device 6A, whereby inflating the inflatable device 6A to secure the BPH tubes 1A-3A to the exterior surface 14 of the first loading tool 4A. Since the loading tool 4A has a larger diameter 17 to its rearward portion 18 than its front portion 19, the larger diameter 17 has a ledge 20 to mate with the BPH tubes 1A-3A at the openings 10-12. While the inflatable device 6A is fixed between the front 19 of the first loading tool 4A, and the ledge 20, the rearward openings 10-12 of the BPH tubes 1A-3A fit the ledge 20. As a result, the front 19 of the first loading tool 4A extends through the front 15 of the BPH tubes 1A-3A, since the front 19 of the first loading tool 4A comprises a pointed portion 21. As a rule, the pointed portion 21 has a system for penetrating an opening defined by the Urethra concerning an entrance about a user's external reproductive organ, whereby the BPH tubs 1A-3A are set within the Urethra to open the Prostate. When the Prostate is opened by use of the BPH tubes 1A-3A, whereby being moved along in Constance frictional contact via the first loading tool 4A, either BPH tube is set in place. Now, the fluids are allowed to flow from the Bladder, through the BPH tubs 1A-3A, and leaving the body via the Urethra. The tool 5A has a pointed end 8A, for penetrating as well. The tool 5A is insulted through either tube 1A-3A, as its end 8A extends thereby.

The Prostate is a male reproductive system for making semen, and for carrying sperm from two testicles to the Prostate via a Seminal vesicle member. The Seminal has a tube joining the Urethra for carrying the sperm through the Seminal tube, three slots 1C, 2C, and 3C about the BPH tubes 1A-3A, and through the Urethra. When at least one BPH tube 3A is set in the Urethra, such that its front surface 15 is in an alignment with the Bladder, its rearward opening 12 is set erectly beneath the Prostate, and the slot 3C is in an alignment with the Seminal tube as well. As the BPH tubes 1A-3A are set in the Urethra via a mid Prostate location for the fluids to flow, the Prostate comprises the Urethra including the BPH tubes 1A-3A, when either tube is set in place. As a rule, the BPH tubes 1A-3A can be left in place within the Urethra for an indefinite time, and without the use of the Foley catheter.

Moreover, the BPH tubes 1A-3A are defined by a system for allowing a user to empty the Bladder accordingly. Anesthesia such as a spinal block is excluded from this procedure, since a conventional clear BPH numbing medication is employed. The installation of the BPH tubes can be performed via the Ultra Sound system, whereby a doctor can use a severe case of scrutiny with respect to the above modification. While the slots 1C, 2C, and 3C each of which is incorporated in the BPH system for receiving sperm from the Seminal tube, the urologist is skilled in the Art, and in such a manner that he/she can set either BPH tube 1A-3A in an alignment with the Seminal tube about either adjacent slot.

Seeing that the aforementioned fluids contain water and waste product, the fluids are made by use of a user's kidneys, stored in the Bladder, and leaves the body via the Urethra. The BPH tubes 1A-3A are installed and set in place by the doctor specializing in the urinary organs in females, and the urinary and sex organs in males. Since the BPH tubes 1A-3A eliminate mild-to-moderate BPH symptoms, medicines, the catheter and surgery, the doctor can perform the installation by use of the Ultra Sound. Because the BPH surgery is unwanted by Millions of American men, the Prostate Cancer is prevented, when the BPH tubes 1A-3A are installed and set in place by way of the ULTRA SOUND. A Flowchart is provided in FIG. 10 as follows: Step 1, apply numbing medication via the Urethra, Step 2, extend element 14 of the loading tool 4A through either tube 1A-3A. Step 3, the pointed portion 21 of the loading tool 4A will penetrate an opening of a reproductive male organ. Step 4, a doctor will insert the portion 21 including the tube via the male organ, and move the tube through the Urethra in opposition to the Bladder by use of the loading tool 4A. Step 5, the doctor will view a monitor of an Ultra Sound, whereby the tube is set 2.0 mm from the base of the Bladder, and 1.0 cm beneath the Prostate. Step 6, the doctor checks the non-surgical procedure via the Ultra Sound. Step 7, the Urethra is protected by the tube as fluids flow via a Seminal tube into a slot of the tube, when a man ejaculates and via the Urethra when he passes water.

While the above description contains many specifics of which should not be construed, as limitations on the scope of the invention, many variations and modifications will thus be apparent to persons skilled in the Art. E.g., Prostate Cancer Symptoms are the same as BPH Symptoms, i.e., trouble passing water, a burning or stinging feeling when passing water, a frequent urge to pass water, especially at night, when there is only a small amount of water, blood in the water, or semen, painful ejaculation, nagging pain in the back, hips or pelvis, and blocked fluids and/or water. As a normal part of aging, the prostate enlarges and can press against the bladder, and the urethra. This can slow down or block water flow. Consequently, this is why a man has trouble passing water, a burning or stinging feeling when passing water, a frequent urge to pass water, and there can be only a small amount of fluids, since in process of time the prostate will be defined by complete blockage. Notwithstanding, the BPH tubes 1A-3A will prevent Prostate Cancer Symptoms by opening the urethra in the area where the prostate is affecting water flow. These tubs 1A-3A each of which can be set in this position to provide water flow, thereby excluding a spinal block, unwanted side effects, and surgery with respect to the above modification. The BPH tubes 1A-3A are installed by a NON-SURGICAL procedure by use of the ULTRA SOUND. Moreover, the slots 1C-3C of the BPH tubes 1A-3A each of which is in an alignment with the Seminal vesicle tube, which is connected to the Urethra. Now, sperm is sent on a short course of travel in a brief space of time, however, through either of the slots 1C-3C, the Urethra, and leaves the body via the reproductive organ, consequently in opposition to a woman's egg, when a man ejaculates.

1. A Benign Prostatic Hyperplasia (BPH) tube in a user's Urethra at the base of the Bladder adjacent to an Enlarged Prostate gland for preventing BPH Prostate Cancer, comprising:
a first conventional tube (1A) having a length starting at said Prostate and ending at said base of said Bladder, thereby receiving fluids via said first tube (1A), said length indicates mild symptoms, said BPH tube prevents symptoms;
a second conventional tube (2A) having a longer length starting at said Prostate, and ending at said base of said Bladder thereby receiving said fluids from said Bladder, said length is defined by moderate BPH symptoms; and
a third conventional tube (3A) having the longest length starting at said Prostate and ending at said base of said Bladder for withdrawing said fluids from said Bladder, when complete blockage occurs, said BPH tube prevents blockage;
a first loading tool (4A) having a conventional inflatable device (6A) for causing stability to said first, second and third BPH tubes (1A-3A) before removing said BPH tubes (1A-3A) from said Urethra, where upon installation, said inflatable device (6A) is deflated;
a second loading tool (5A) having an exterior threaded surface (7A) at its front end (8A), and engaging three interior threaded surfaces (9A) in three rearward openings (10-12) of said BPH tubes (1A-3A), whereby to load, or unload said BPH tubes (1A-3A) through said Urethra;

2. A BPH tube for being loaded and unloaded via said Urethra as defined in claim 1, wherein said BPH tubes (1A-3A) are shaped interiorly to fit the exterior surface (14) of said first loading tool (4A), said first loading tool (4A) extends through the front (15) of said BPH tubes (1A-3A).

3. A BPH tube for being loaded and unloaded via said Urethra as defined in claim 1, wherein said first loading tool (4A) has a hole (16) below said inflatable device (6A) for inflating said inflatable device (6A) to secure said BPH tubes (1A-3A) to said first loading tool (4A).

4. A BPH tube for being loaded and unloaded via said Urethra as defined in claim 1, wherein said first loading tool (4A) has a larger diameter (17) at its rearward portion (18) than its front portion (19), said larger diameter (17) has a ledge (20) to mate with said BPH tubes (1A-3A) at said openings (10-12).

5. A BPH tube for being loaded and unloaded via said Urethra as defined in claim 1, wherein said inflatable device (6A) is fixed between the said front (19) of said first loading tool (4A) and said ledge (20), said rearward openings (10-12) of said BPH tubes (1A-3A) fit said ledge (20).

6. A BPH tube for being loaded and unloaded via said Urethra as defined in claim 1, wherein the said front (19) of said first loading tool (4A) extends via the said front (15) of said BPH tubes (1A-3A), the said front (19) of said first loading tool (4A) has a pointed portion (21).

7. A BPH tube for being loaded and unloaded via said Urethra as defined in claim 6, wherein said pointed portion (21) has a system for penetrating an opening about said Urethra, said BPH tubes (1A-3A) are set within said Urethra to open said Prostate.

8. A BPH tube for being loaded and unloaded via said Urethra as defined in claim 1, wherein said Prostate gland is opened via said BPH tubes (1A-3A), said fluids are allowed to flow from said Bladder through said BPH tubes (1A-3A), and via said Urethra.

9. A BPH tube for being loaded and unloaded via said Urethra as defined in claim 1, wherein said Prostate is a male reproductive system for making semen, and for carrying sperm from two testicles to said Prostate via a Seminal vesicle member.

10. A PBH tube for being loaded and unloaded via said Urethra as defined in claim 9, wherein said Seminal has a tube joining said Urethra for carrying said sperm through said Seminal tube, three slots (1b-3b) and through said Urethra.

11. A PBH tube for being loaded and unloaded via said Urethra as defined in claim 1, wherein at least one BPH tube (3A) is set in said Urethra having its front surface (15) near said Bladder, its rearward opening (12) set erectly beneath said Prostate gland, and said slot (3b) is adjacent to said Seminal tube.

12. A PBH tube for being loaded and unloaded via said Urethra as defined in claim 11, wherein said BPH tubes (1A-3A) are set in said Urethra via a mid Prostate location for said fluids to flow, said Prostate comprises said Urethra including said BPH tubes (1A-3A).

13. A PBH tube for being loaded and unloaded via said Urethra as defined in claim 12, wherein said BPH tubes (1A-3A) are left in place via said Urethra for an indefinite time, and without the use of a Foley catheter.

14. A PBH tube for being loaded and unloaded via said Urethra as defined in claim 12, wherein said PBH tubes (1A-3A) are defined by a system for allowing a user to empty said Bladder accordingly.

15. A BPH tube for being loaded and unloaded via said Urethra as defined in claim 14, wherein said PBH tubes (1A-3A) are set in said Urethra via a clear BPH numbing medication, said installation is viewed via a monitor of an Ultra Sound.

16. A BPH tube for being loaded and unloaded via said Urethra as defined in claim 10, wherein said slots (1b, 2b, 3b) are incorporated in said first BPH tube (1A), said second BPH tube (2A), and said third BPH tube (3A) for receiving said sperm from said Seminal tube, said Ultra Sound reveals said tube via said slots.

17. A BPH tube for being loaded and unloaded via said Urethra as defined in claim 12, wherein said fluids containing water and waste products, said fluids are made by a user's kidneys, stored in said Bladder and leaves the body via said Urethra.

18. A BPH tube for being loaded and unloaded via said Urethra as defined in claim 17, wherein said BPH tubes (1A-3A) are installed and set in place by a doctor specializing in the urinary organs in females, and the urinary and sex organs in males.

19. A BPH tube for being loaded and unloaded via said Urethra as defined in claim 1, wherein said BPH tubes (1A-3A) eliminate mild-to-moderate BPH symptoms, Medications, said catheter and surgery, when said BPH tubes (1A, 2A or 3A) are set in place, said doctor is performing said installation via said ULTRA SOUND.

20. A BPH tube for being loaded and unloaded via said Urethra as defined in claim 19, wherein said BPH surgery is unwanted by Millions of American men, said Prostate Cancer is prevented, when said BPH tubes (1A-3A) are installed and set in place by the use of said ULTRA SOUND.
